(19) United States
(12) Patent Application Publication
(10) Pub. No.: US 2024/0226060 A1
(43) Pub. Date: Jul. 11, 2024

(54) TOPICAL LIPOSOME POLYPHENOL COMPOSITIONS FOR TREATING AND PREVENTING VARIOUS SKIN DISORDERS AND METHODS OF PREPARATION THEREOF

(71) Applicant: PANAG PHARMA INC., Halifax (CA)

(72) Inventors: Donald L. Simmons, Dollard-des-Ormeaux, QC (CA); B. Matthew Allain, Shad Bay, NS (CA); Melanie Kelly, Halifax, NS (CA)

(21) Appl. No.: 18/558,909

(22) PCT Filed: May 3, 2021
(86) PCT No.: PCT/CA2021/050614

§ 371 Date: Nov. 3, 2023

(51) Int. Cl. A61K 31/353; A61K 9/00; A61K 9/06; A61K 9/127; A61K 9/48; A61K 31/00; A61P 17/10

(52) U.S. Cl. CPC A61K 31/353; A61K 9/0014; A61K 9/06; A61K 9/127; A61K 9/4833; A61K 31/658; A61P 17/10

(57) ABSTRACT

Liposomal polyphenol compositions comprised of lipophilic glycoside free flavonoids and cannabinoids, each separately and optimally encapsulated within membrane lipid bilayers of mostly spherical vesicles, their methods of preparation and their potential use when dispersed into pharmaceutically acceptable bases for treating and preventing various skin disorders.

### FIELD OF THE INVENTION

This invention relates to liposomal polyphenol compositions comprising a therapeutically effective amount of a lipophilic glycoside free flavonoid or a lipophilic glycoside free cannabinoid optimally encapsulated within the membrane lipid bilayers of spherical vesicles, and methods for making thereof.

The invention also relates to inclusion of these compositions into pharmaceutical acceptable bases and the use of such final products to help prevent and treat reddened itchy dry skin conditions caused by environmental stress exposures such as solar radiation and atmospheric pollutants.

The uses also extend to treatment of symptoms of inflammatory dermatoses involving epidermal and dermal skin tissues. These dermatoses include but are not limited to, atopic dermatitis, hyperpigmentation, contact dermatitis, allergic dermatitis, aural eczematoid dermatitis, otitis externa, acne, as well as painful inflammatory skin conditions arising as a result of genetic, immune or neuropathic disease including, but not limited to, epidermolysis bullosa, bullous pemphigold, pyoderma gangrenosum, sarcoidosis, scleroderma, cutaneous mastocytosis, postherpetic neuralgia.

### BACKGROUND OF THE INVENTION

Flavonoids and cannabinoids each possessing polyphenol groupings are the major focus of this disclosure due to their significant health benefits as exhibited through recent clinical publications. These benefits were obtained with conventional topical dosage forms with inherent instability problems of these polyphenols when dispersed in aqueous systems or left exposed to normal atmospheric conditions. A classic example of the latter problem is cutting an apple in half and leaving the two cores exposed to the environment. Within a very short period of time the two cores turn light brown indicating an oxidative reaction involving catechin and quercetin, two polyphenolic flavonoids present in apples. Flavonoids are biologically active and beneficial dietary ingredients that are ubiquitous in nature and found in fruits, vegetables, tea and cocoa. Most flavonoids occur in plants as water-soluble β-glycosides, i.e., bound to one or more sugar molecules. The glycoside free and lipophilic flavonoids are polyphenolic compounds that are categorized according to chemical structure primarily into six groups, i.e., flavones, flavonols, flavonones, anthocyanidins, isoflavones, flavanols. Their basic structure consists of two aromatic rings A and B each containing one or more phenolic groupings and the two rings linked by a 3-carbon chain forming a closed pyran ring C containing oxygen. Numerous subgroups exist within these groups and are gaining widespread attention in the health care industry. Some current well-documented flavonoids are epigallocatechin gallate (EGCG), silybin, quercetin, apigenin and kaempferol that are isolated from a variety of plants, vegetables and fruits. The latter three flavonoids are also congregated (Izzo et al, Molecules 2020 25, 631) in the plant Cannabis sativa (C. sativa) along with four cannabinoids of equal therapeutic interest namely cannabidiol (CBD), cannabigerol (CBG), cannabinol (CBN) and tetrahydrocannabinol (THC). Also notable is the presence of the resorcinol or 1,3-benzene diol component in all of these molecules, including their related analogs. Topical products containing pure resorcinol were developed during the late 19^{th} century primarily as a keratolytic to treat corns, calluses and warts but are still available to treat acne, seborrheic dermatitis, psoriasis and other skin disorders. The recent entry of flavonoid and cannabinoid products into the personal care market have obviously reduced the more serious adverse events associated with resorcinol.

Cannabinoids, such as cannabidiol (CBD), cannabigerol (CBG) and cannabinol (CBN), have received significant scientific and medical attention recently due to their potential therapeutic benefits and absence of behavioral side-effects (Burstein, Biorganic & Medicinal Chemistry 2015 23:1377-85). All three compounds including several of their analogs, namely cannabidiolic acid (CBDA) and cannabidivarin (CBDV), are examples of cannabinoids like flavonoids, that rely on the resorcinol moiety for the major biological activity contribution by the molecule. They differ from flavonoids in that common attachments to the resorcinol moiety are unsaturated hydrocarbon chains called terpenes, including their oxygenated-counterparts, terpenoids. The therapeutic potential of these cannabinoids arises from their interactions with the endocannabinoid system (ECS), a complex cell-signaling system in the human body that is responsible for the modulation of critical biological functions including neurotransmission, immune response, pain and metabolism (Lu, Mackie, Biological Psychiatry 2016 79(7):516-525). The ECS has two primary receptors: CB1 receptors that are extensively expressed in the central nervous system and CB2 receptors, which are mostly found in the peripheral nervous system, especially immune cells. CBD, along with the pyschoactive phytocannabinoid THC, can interact with CB1 receptors located throughout the body and can modify the activity of cannabinoid ligands that bind to CB1, such as THC. The anti-inflammatory and analgesic effects of CBD may arise in part from interactions with the CB2 receptor, as well as other non-cannabinoid receptors (Larsen, Shahinas, Journal of Clinical Medical Research 2020 12(3):129-141). Experimental and human studies report that CBD reduces pain sensation and additionally acts as an anxiolytic to decrease anxiety and can reduce seizure activity in certain seizure disorders.

CBG, a precursor to THC and CBD, is considered a more minor presence in C. sativa compared to THC and CBD. CBG activates both CB1 and CB2 receptors, as well as non-cannabinoid receptors such as peroxisome proliferator activated receptor gamma (PPARγ), a member of the nuclear steroid receptor family (Pertwee et al, Pharmacological Reviews 2010 62:588-631). CBG has demonstrated anti-inflammatory, antioxidant and antibacterial actions and combinations of CBG and CBD were shown to be neuroprotective in both in vitro and in animal models, underscoring the therapeutic potential of CBG and CBD-CBG combinations (Mammana et al, Medicina 2019 55, 747). CBN is a non-intoxicating cannabinoid that is isolated from aged Cannabis plants and is associated primarily with a change in the chemical structure of the THC molecule. Neuroprotective activity was also displayed by CBN in a rodent model (Weydt P et al, Amyotrophic Lateral Sclerosis and Other Motor Neuron Disorders 2005 3:182-184). Such potential may ultimately be recognized in reducing severity of neurological illnesses such as Huntington's disease (HD), amyotrophic lateral sclerosis (ALS, more commonly known as Lou Gehrigs' disease), Parkinson's disease (PD) and multiple sclerosis (MS).

In plants, flavonoids and cannabinoids perform numerous functions such as providing colour and scent to fruits, flowers and vegetables while primarily contributing protection to the plants from the same environmental pollutants affecting humans, i.e., solar UV radiation, reactive oxygen species, insects, harmful microbes, etc. Over 4,000 flavonoids and 140 cannabinoids have been isolated and characterized to date and provoked considerable interest because of their potential beneficial effects on human health. These benefits have been identified as antioxidant, anti-inflammatory, antimicrobial, antiviral, analgesic, muscle relaxant, anti-carcinogenic, etc. Botanical extracts containing flavonoids and cannabinoids have been utilized as medicines dating back millennia.

### Conventional Topical Formulations—Clinical & Pre-Clinical Evidence

The use of pure glycoside free lipophilic flavonoids in successfully treating several common skin diseases has been reported in recent medical literature. Of particular interest is the use of relatively low concentrations (1%) required to accomplish these clinical successes and we believe is indicative of the relatively high potency accorded these ingredients. Suh et al (Journal of Investigative Dermatology, 2013 133:429-440) demonstrated a significant improvement in facial acne vulgaris of patients using pure epigallocatechin 3-gallate (EGCG) from green tea leaves. Hydroalcoholic solutions containing 1 and 5% EGCG were randomly allocated to one half side of the face while the opposite side was treated with vehicle control (3% alcohol). Global assessment of acne severity was performed using the revised Leeds score that reflects both inflammatory (papules, pustules, nodules, cysts) and noninflammatory acne lesions (comedones). At week 8, revised Leeds scores for the 1 and 5% EGCG were significantly decreased from a 5.1 baseline value to 1.2 and 1.7, respectively. Mean noninflammatory and inflammatory lesion counts for 1% EGCG were significantly decreased by EGCG to 79 and 89% respectively, when compared to baseline and vehicle control after only 8 weeks EGCG application. The 5% EGCG formulation showed a parallel improvement in acne lesion counts to the 1% formulation with no clinically significant differences between the two treatments. The authors concluded that EGCG topically applied at 1% concentration can directly impact three out of four pathological processes in acne including excessive sebum production, inflammation and Propionibacterium acnes proliferation. Of special interest is the 8-week time interval required in the treatment when compared to 12 weeks normally required for prescription drug acne therapy.

A second flavonoid, silybin from the milk thistle plant, displayed exceptional efficacy in treating melasma. In a study involving 96 patients, Altael (BMC Dermatology 2012 12(18):1-6) conducted a double blind, randomized evaluation of silymarin creams (0.7% and 1.4%) versus a placebo cream. Silymarin is a mixture of several structurally-related flavonoids but the major and most active component is silybin. Formulations were applied topically to affected areas twice daily for 4 weeks. Treatment response as rated by size of lesions was performed weekly along with skin pigment evaluation according to melasma area severity index (MASI). The results demonstrated significant size reduction of lesions by the silymarin creams at the end of the 1st week compared to placebo with complete clearing of lesions by the active creams at the 4th week. MASI scores demonstrated a reduction of 100% after 1 month! Somewhat different treatment results were obtained by Nofal et al (Journal of Cosmetic Dermatology 2019 18(1):263-270) who compared efficacies of 0.7% and 1.4% silymarin creams and 4% hydroquinone cream in melasma patients. MASI scores were significantly reduced from baseline values in all groups, e.g., reduction values of 36 and 34% for silymarin 0.7 and 1.4% respectively, and 39% for hydroquinone but only after three months! Zero side effects were recorded with silymarin creams while hydroquinone was associated with 71% of patients presenting with erythema, burning and scaling. Like the EGCG study cited above, no clinically significant differences were observed between the 0.7 and 1.4% silymarin formulations thus demonstrating again that treatment benefits of flavonoids applied to skin disorders can be achieved at concentrations of 1% or less.

Quercetin in the form of quercetin-phytosome (1%) was successfully used in a monocentric and single blind trial to treat four trauma-induced skin episodes in 30 human volunteers (Maramaldi et al, Clinical Cosmetic Investigative Dermatology 2016 9:55-62). The results showed a significant soothing effect against skin inflammation induced by UV radiation, histamine prick test and skin barrier disruptions induced by the acknowledged skin irritants sodium lauryl sulfate (SLS) and gallic acid solutions. Treatment efficacy was exhibited by reductions in redness (erythema), itching and inflammation of damaged skin. The results also showed quercetin's anti-allergic properties by the reduction in irritation and itching caused by histamine prick test. Of special interest is the fact that quercetin content in the phytosome (Quercevita™, Givaudan Active Beauty) ranges between 17-22% so the actual quercetin concentration is ˜0.2 percent. An additional critique is that the placebo cream used in the study had water only replacing 1% Quercevita™ resulting in removal of both quercetin and phospholipids. The mild-to-moderate anti-inflammatory activity of phospholipids is documented in the scientific literature so its treatment contribution in the placebo may be significant. A similar concentration of quercetin encapsulated in liposomes may be an interesting product for the pediatric marketing segment.

In a randomized and double-blinded anti-aging study conducted in forty women (ages >30 years), Choi et al (International Journal of Molecular Medicine 2016 38:627-634) used 1% apigenin in a cream formulation versus placebo cream. The formulations were used on facial applications, including eye rim, to monitor skin elasticity, dermal density, length of crow's feet, skin moisture, transepidermal water loss (TEWL) and facial evenness. Applications were performed twice daily with clinic visits scheduled at 2 and 4 weeks to perform chosen skin measurements. Significant improvements were found in dermal density, skin elasticity, reduction in length of fine wrinkles (especially crow's feet) after 2 and 4 weeks of subjects using apigenin-containing cream. The study also demonstrated that skin moisture content, TEWL and skin texture were all significantly improved after 2 and 4 weeks of apigenin use. The authors concluded that topical apigenin application reduces aging phenomena and its aging-associated clinical signs. These low dosage requirements for flavonoids to obtain clinical successes in skin disease therapy combined with their low toxicity profiles provides a significant new therapeutic entry into the arena of cosmetic medicines, i.e., cosmeceuticals and dermatologicals.

Like flavonoids, a most common recommendation for cannabinoid concentration in skincare formulations appears to be around 1%. Unlike flavonoids however, clinical data on topical cannabinoid treatment is lacking to date and in vivo activity studies confined mostly to animal data. However, we believe a great deal of similarity exists between them when comparing dosage requirements. Westlund et al (European Journal of Pain 2016 20(6):936-94) evaluated carbomer-gel formulations containing 0.1, 0.5, 1 and 10% CBD in alleviating swelling and pain in arthritic-pain induced Sprague-Dawley rats. To induce arthritis, animals were anesthetized and one knee joint injected with complete Freud's adjuvant (CFA) emulsion. Animals were returned to individual cages and monitored daily. Joint circumference and pain-related behaviours were assessed prior to CFA injection and daily beginning on day 3 after CFA treatment (days 3-7). The gels were applied to shaved backs of the animals for 4 consecutive days following arthritis induction. Results demonstrated that the 1% dose optimally reduced swelling and alleviated pain but increasing dosage to 10% did not yield additional improvement. Lower doses of CBD had no effect on CFA-induced edema but the authors commented that results may have been different if the gels had been directly applied to the wound sites instead of animal backs.

Medical groups from Stanford and West Virginia Universities reported on the self-initiated use of topical cannabidiol in treating epidermolysis bullosa (EB) in 3 patients (Teng et al, Pediatric Dermatology 2018 35:e224-e227) whose ages were 6 months, 3 years and 10 years. EB is a rare blistering skin disorder that is challenging to manage because skin fragility and repeated wound healing cause itching, pain, limited mobility, and recurrent infections. All 3 patients initiated treatment with CBD oil without physician recommendation. One patient was weaned completely off oral opioid analgesics. All 3 reported faster wound healing, less blistering, and amelioration of pain with CBD use. The authors concluded that although the results demonstrated promise, further randomized, double-blind clinical trials are necessary to provide scientific evidence of CBD for the treatment of epidermolysis bullosa.

In a similar study, Maida and Corban (Journal of Pain and Symptom Management 2017 54:732-736) reported on the use of combined CBD and THC in treating pyoderma gangrenosum, i.e. leg ulcers, in 3 elderly patients. Prior to treatment with combined CBD and THC (each less than 1%) in sunflower oil, two patients had used oral and intralesional steroids plus oral opioids (morphine sulfate) and the third patient both oral steroids and acetaminophen up to 650 mg every 6 hours to treat excess pain. Patient number one dosage consisted of 0.5% THC and 0.7% CBD, a balanced combo from Cannabis indica, whereas the remaining two patents received dosages of 0.7% THC and 0.9% CBD, a combo from Cannabis sativa. All patients reported pain relief within three to five minutes of applying the topical treatments. Researchers followed pain scores on a scale of 0 to 10, both pre- and post-treatment for a varying number of weeks. Scores for the two patients on opioids were highly significant, e.g., pre-treatment 8.25 and 8.75 and post-treatment 2.76 and 2.33 with corresponding p values of 0.0007 and 0.0006, respectively. Opioid usage decreased from 26 mg to 0.24 mg in one patient and from 27.3 to 12.5 mg in the second patient. The third patient recorded scores of 4.29 and 1.50. Analgesic effects were indeed impressive according to the authors but larger more convincing research (double-blind, randomized, controlled trials) into the pain-relieving effects of topical cannabinoids are necessary.

### Conventional Topical Formulations—Stability Concerns

A major concern for scientists in new product development of topical flavonoid and cannabinoid formulations however is the instability of these oxygen-sensitive bioactive ingredients. The stability of 0.3% hesperetin glycoside (HG) was investigated along with similar concentrations of its phenol-esterified counterpart in standard oil/water emulsions with pH 5.5 values and storage at 40° C. for 120 days (Vertuani et al, Cosmetics 2018 5(4),72). The esterified derivatives were relatively stable at the end of the 120-day period but the unesterified HG sample underwent a color change from yellow to brown after 30 days with an ingredient content of only 16.7% at the end of the study. Similar flavonoid instability problems were circumvented by scientists in clinical and preclinical investigations by paying special attention to duration of treatment, type of packaging container and storage conditions of the clinical samples. In a clinical study involving silymarin cream in melasma patients, Elfar et al (Journal of Clinical & Experimental Dermatology Research 2015 6(3):1-7) recognized a need for extra precautions in their silymarin cream clinical samples by utilizing airtight containers and delivering freshly prepared formulations to patients each week for the entire period of twelve weeks.

A similar problem with flavonoid cream stability was encountered by Katiyar et al (Neoplasia 2003 5(6):555-565) in a long term anti-photocarcinogenic study involving EGCG dispersed in a hydrophilic cream for use in a hairless mouse model. The cream formulation was prepared on a weekly basis and stored at 4° C. These instability concerns are readily circumvented by optimal encapsulation of these and other lipophilic flavonoids, including cannabinoids, within the membrane lipid bilayers of multilamellar vesicles. Like flavonoids, cannabinoid stability is influenced by temperature, oxygen and light. Torres-Suarez et al (Journal of Chromatography B 2020 May 22, Article 122188) utilized HPLC to evaluate CBD stability under various storage conditions. CBD loss when dispersed in simulated physiological conditions (pH 7.4 and 37° C.) was 10% within 24 hours. The authors concluded that CBD is highly unstable and this should be a prime factor in the development of pharmaceutical dosage forms.

### Drug Encapsulation in Topical Formulations

The pioneer in adapting multilamellar lipid vesicles (MLVs) to topical therapeutic applications was the late Professor Michael Mezei of Dalhousie University in Halifax Nova Scotia Canada. His initial patent (U.S. Pat. No. 4,485,054) focused on encapsulation efficiency of lipid soluble medicaments intended to produce local (i.e., topical) rather than systemic action with emphasis on the steroid hormone, progesterone. Utilizing a modified version of the thin film hydration method, a formulation containing dipalmitoyl phosphatidylcholine (DPPC) 22.2 mg, cholesterol 5 mg, progesterone 5 mg (labelled) and 5 mL 8 mM calcium chloride solution was found to possess an encapsulation efficiency (EE) of 77 percent for progesterone. The relatively high cholesterol to phospholipid ratio (˜1:4) used in the formulation appeared to have had a negative impact on the progesterone encapsulation efficiency. Since cholesterol possesses a similar structure to progesterone, a competition for bilayer spatial sites is not unexpected. Deniz et al (Bioscience Reports 2010 30:365-373) also utilized the thin film hydration method to evaluate distearoyl phosphatidylcholine (DSPC) and the effect of different cholesterol to DSPC ratios, e.g., 0, 1:2, 1:5, 1:10, on encapsulation efficiency of the lipophilic drug celecoxib. A cholesterol-free formulation followed closely by the 1:10 and 1:5 formulas provided the optimum EE values whereas a significant decrease was encountered at the 1:2 ratio.

Subsequent patents and publications by Mezei were used to demonstrate significantly greater drug concentrations in animal epidermis and dermis skin sites from application of drugs encapsulated in MLVs than from the same drugs applied from conventional topical formulations. Conversely, significantly higher urinary drug content was found with the conventional formulations. In U.S. Pat. No. 4,897,269, Mezei investigated 2% minoxidil encased in MLVs, in suspension (identical ingredients and quantities used in MLVs) and in solution applied to shaved dorsal areas of albino guinea pigs twice daily for 3 days. Liposome & suspension each contained solvents ethanol (10%) & propylene glycol (7%) and 1:2 cholesterol:lipid ratio whereas the solution contained 60% alcohol and 20% PG (a 2% minoxidil solution formulation prepared according to a brand product (Rogaine® Johnson & Johnson Inc). Skin samples were obtained 4 hours after last dose and cumulative urine samples at study end. The results showed significant increases in drug content samples from skin surface (3-4 fold), epidermis (2-3 fold) and dermis (4-5 fold) with the liposome formulation versus suspension & solution. Conversely, significant less drug was obtained from liposome urine samples.

Similar biodistribution results were later confirmed in U.S. Pat. No. 9,775,789 using single daily applications of liposomal clobetasol propionate (CP, tritium-labelled) formulations (0.025 & 0.05% CP) and a brand product containing 0.05% CP (Dermovate® Cream, Glaxo) applied to the dorsal skin of hairless guinea pigs. Combined epidermis-dermis values for 0.025% CP liposome and Dermovate provided similar cutaneous clobetasol values whereas the 0.05% CP liposome formulation provided more than double the amount of drug. Cumulative fecal and urinary drug outputs after 4 days were ˜7 fold greater for Dermovate versus corresponding liposomal formulations. Propylene glycol is a non-medicinal ingredient present in Dermovate but the drug concentrations found in urine and fecal samples in this study suggests a relatively high content of this acknowledged permeation enhancer.

Much controversy still exists in the scientific community on how these MLVs are able to transport drug into and through the skin's stratum corneum. A general consensus is that MLVs do not penetrate intact into the skin. One possible mechanism is that MLVs after skin application collapse into bilayer sheets that adhere to the surface of the stratum corneum (SC) where the phospholipids and accompanying solvents interact with the SC bilayer lipids to form open channels. The lipid sheets then function as reservoirs to monitor drug release through these SC channels into deeper layers of the skin. This skin reservoir concept has support from the Mezei minoxidil study cited above where the drug remaining on the skin surface following the liposome application was 3-4 times the amount found following suspension and solution formulations.

Several important modes of action are proposed to occur following the collapse of the MLVs: a). solvent-promoted fluidization of SC lipids; b). solvent-promoted permeation and interaction of the liposome lipid components phospholipid and cholesterol with SC lipids, and; c). solvent-promoted permeation of drug by osmotic action into and through SC channels to epidermal and dermal sites while limiting drug transport into the body's circulatory system. Liposomes are therefore considered to be both drug transporters and localizers. Careful attention should be addressed to solvent content however since excessive concentrations can lead to undesirable skin adverse events and possible greater drug entry into the circulatory system. The latter possibility was definitely evident in the animal biodistribution study involving Dermovate cited above and also the time limits set on its use in treating a variety of skin conditions in humans.

Bouwstra (Drug Discovery Today: Technologies, 2005, 2(1):67-74) discussed vesicles as a tool for transdermal and dermal delivery. Bouwstra further recognized the many advantages of skin as a target for drug delivery and attributed the limited number of transdermal drugs on the market at the time to the limited permeability of drugs due to the stratum corneum. A contributing factor is the importance of vesicle physicochemical properties on drug permeation and these properties have been demonstrated in studies showing the moderately enhanced drug transport achieved with liquid state vesicles (unsaturated lipid side chains) versus gel state vesicles (saturated lipid side chains). Numerous attempts have been made by scientists to disrupt and weaken the highly organized intercellular lipids of the stratum corneum in an attempt to either enhance drug transport across the intact skin or to increase the driving force for permeation of drugs across this skin barrier. In the absence of liquid state vesicles, numerous penetration enhancers have been used by scientists such as propylene glycol, alcohol, azone, dimethyl sulfoxide, etc. Propylene glycol has been used as a penetration enhancer for years in many high potency corticosteroid-containing creams and ointments in order for drug to reach dermal vasculature sites, a prerequisite in psoriasis therapy.

Several of the more recently referenced transdermal delivery systems are categorized as elastic or deformable vesicles and referred to as transfersomes and ethosomes. The former system developed by Cevc (U.S. Pat. Nos. 7,175,850, 7,473,432) utilizes lipids and various non-ionic surface active agents or surfactants such as polysorbate 80 to transport corticosteroids and non-steroidal anti-inflammatory (NSAIDs) drugs into the deeper skin layers. The surfactants, referred to as edge activators, are utilized to soften the bilayers of the resulting uni- and bi-laminar vesicles. A shortcoming of these vesicles is the difficulty of loading hydrophobic drugs into the vesicles without compromising their deformability and elastic properties. An additional problem is the near neutral pH values (6.4-8.3) used in their examples, values which would be detrimental to stability of polyphenols. Similar surfactants were utilized to encapsulate cannabinoids (U.S. Pat. No. 10,709,748) in micelles having uni-, oligo- and multi-lamellar structures. The encapsulation efficiencies for cannabidiol (CBD) and tetrahydrocannabinol (THC) were found to be 86% and 92%, respectively. The pH 7.0 selection for these latter formulations is a definite concern for a company hoping to achieve a 2 year shelf life for any product resulting from these formulations.

Ethosomes were developed by Touitou (U.S. Pat. Nos. 5,540,934, 5,716,638) for both transdermal and dermal delivery of various cosmetic and pharmaceutically active drugs. The compositions are comprised of phospholipids (0.5-10%), ethanol or isopropyl alcohol with and without propylene glycol (total organic solvent of 40%) and at least 20 percent water. A high drug content (10%) relative to phosphatidylcholine content (5%) suggests a very low encapsulation efficiency for the drug and a major stability problem if the lipophilic drug is a flavonoid or cannabinoid. Mezei also used high solvent concentrations of both alcohol (10%) and propylene glycol (7%) in all his US Patents, including 1:2 and 1:4 cholesterol to phospholipid ratios in his examples. A major concern is the enhanced potential for skin irritancies with the high concentrations of both solvents and phospholipids proposed for these formulations.

The strong association between phospholipids and lipophilic flavonoids may be a result of the relatively high encapsulation efficiencies achieved during liposome preparation. Both ethanol and propylene glycol alone or in combination have been used as solvents in liposome preparation. An additional factor in achieving high encapsulation efficiency values may be the relatively low flavonoid to phospholipid ratio (˜1 to 10) used in these studies. Quercetin-loaded liposomes prepared by Shaji et al (Asian Journal of Pharmaceutics 2012 6(3):218-226) using the film hydration method achieved 82.6% quercetin encapsulation efficiency. EGCG-loaded liposomes prepared by Ulrih (Journal of Science and Food Agriculture, 2016 96:4623-4632) using a proliposome method, yielded an encapsulation efficiency of 97.5%. Stability studies were conducted on the latter liposomes at 25° C. with pH values set at 2, 4 and 6. The results demonstrated that EGCG was most stable at pH 4 and least stable at pH 2 and 6. The authors commented that their data was consistent with that of previous authors who reported that the highest stability of catechins was from pH 4.0 to 5.2.

El-Khordagui et al (AAPS PharmSciTech 2012 13(2):723-731) employed a variation of the proliposome method using propylene glycol usage instead of alcohol to achieve an encapsulation efficiency of 95.6% for miconazole nitrate. Such high encapsulation efficiencies are not unexpected especially when the amount of lipophilic flavonoid or cannabinoid compared to phospholipid is in the 10% range as in the above cited studies. Differences in encapsulation efficiencies are seen however between a lipid flavonoid and its glycoside counterpart (Demetzos et al, Journal of Pharmacy and Pharmacology, 2004 56:1217-1224). Using isoscutellarein or its glycoside counterpart incorporated in liposomes at a final molar ratio of 9 parts egg PC to 2 parts flavonoid, the authors found an incorporation efficiency of 95% for isoscutellarein and only 37.5% for the glycoside. The values are not expected to be due to molar mass differences, e.g., 286 g/mole versus 448 g/mole for glycoside, but the possible exclusion of cholesterol in the formulation which would help prevent leakage of the slightly more water-soluble glycoside into the surrounding aqueous medium.

In U.S. Pat. No. 9,775,789, a production scale-up process is described for encapsulation of <0.25% polyphenolic ingredients into multilamellar liposomes. The technology consisted of adding purified water to a mixture of glycerophospholipid, polyphenolic agent and trace amounts of skin-related lipids pre-dissolved in ethyl alcohol. The mixing of the two solutions were conducted at a temperature above the transition temperature (>55° C.) of the phospholipid used in the process. The end result was a creamy white liposome dispersion. The polyphenol used in the disclosure was the flavonoid epigallocatechin 3-gallate (EGCG) at 1-part EGCG to 10-part phospholipid ratio as in previously cited examples. Efforts to include the total required EGCG amount (0.5%) in the predetermined upper limit of ethyl alcohol (3%) was not possible because of the increased content of both flavonoid and phospholipid required in the formulation. In order to meet a final product label claim, additional EGCG was included in the base gel-cream portion of the final topical composition to coincide with Mezei's patented multiphase liposome specifications, i.e., drug encapsulated in liposomes, drug dissolved in aqueous channels and surrounding medium and drug in solid suspended form.

Extensive discoloration occurred however in this final composition after relatively short-term storage at room temperature in 40-gram laminate tubes, thus indicating the need for optimal encapsulation of lipophilic polyphenols into membrane bilayer lipid vesicles. Total encapsulation effect on stability enhancement became visibly evident on a liposomal EGCG dispersion sample (representing 0.20% EGCG content) retained without nitrogen blanketing and under refrigeration for over four years. The sample had been removed to RT conditions on numerous occasions for visual examination and returned with minimal disturbance to original storage conditions. Ultimately the sample had separated into two distinct phases: a clear colorless transparent supernatant phase on top and a pearly white sediment on the bottom. This total lack of discoloration provided evidence of a physically stable formulation, but chemical stability confirmation obviously required through HPLC or spectrophotometric analyses.

Proper solvent selection is also an important factor in drug encapsulation since some low molecular weight lipophilic flavonoids such as apigenin (270 g/mole) possess poor solubility performances in solvents such as ethanol but superior solubilities in propylene glycol and polyethylene glycol 400 (Shakeel et al, Journal of Molecular Liquids 2017 234:73-80). In addition to ethyl alcohol, propylene glycol may be selected as co-solvent contributor. An upper limit of about four percent content is selected for each solvent contribution in both the flavonoid or cannabinoid compositions added to the final formulation. The solvent specifications are easily attainable due to the relatively low concentrations of lipophilic polyphenols (1.0%) required for treating skin disorders. If necessary, additional skin penetration enhancers can be incorporated within the aqueous phase of the pharmaceutically acceptable base. The additional processing step should not be necessary due to the increased specifications for each solvent (about 4%) since most projected encapsulation requirements will be in the 0.5% to 0.75% content range.

High purity phosphatidylcholines (PC) with saturated side chains are preferably used because of their lack of irritation potential, proven moisturizing capability and moderate complementary anti-inflammatory activities. Of special significance is their ability to provide thicker bilayer values than their corresponding unsaturated side chain counterparts. Bilayer thickness values for 1,2-dipalmitoyl (DPPC) and 1,2-distearoyl (DSPC) at 25° C. were found to be 4.28 and 4.7 nm (Sun et al Biophysics Journal 1996 71:885-891). Pure hydrogenated phosphatidylcholine (HPC) with both palmitoyl and stearoyl side chains is estimated to have a bilayer thickness value at 25° C. between those of DPPC and DSPC, i.e., 4.5 nm. In similar experiments conducted at a higher temperature, Katsaras et al (Soft Matter 2016 12(47):9417-9428) reported bilayer thickness values at 50° C. for fully hydrated fluid bilayers of 1,2-dilauroyl (DLPC), 1,2-dimyristoyl (DMPC) and 1,2-dipalmitoyl (DPPC) of 2.96, 3.22 and 3.86 nm, respectively. These ingredients represent phospholipids with 12, 14 and 16 saturated carbon bilayers, respectively. The authors also demonstrated that the inclusion of double bonds in the side chains results in a significant thinning of the lipid bilayer. For example, the bilayer thickness at 30° C. of 1,2-diarachidonoylphosphatidylcholine (DAPC), a phospholipid comprised of two polyunsaturated arachidonic acid chains each with 20 carbons and 4 double bonds, had a bilayer thickness of 2.98 nm, or identical to that of the dilauroyl or 12 saturated carbon phospholipid (DLPC). This reduced spatial environment for liquid state vesicles may negatively impact the optimal encapsulation efficiency of some lipophilic flavonoids, especially those possessing slightly larger molar masses. An additional concern is exposure of the double bonds in the phospholipid side chains to oxidation, thus resulting in the creation of harmful peroxides.

There is thus a need for preparation of optimally encapsulated polyphenol compositions using conventional cosmetic manufacturing equipment such as jacketed stainless-steel containers, propeller stirrers, peristaltic pumps, etc. Such compositions are then easily dispersed into pharmaceutically acceptable cream, lotion, gel or suspension formulations. Production scale-up operations have already been achieved, for example a 200 Kg batch of a liposome placebo cream used in a previous clinical trial involving 0.5% titrated extract of Centella asiatica or TECA (U.S. Pat. No. 9,775,789), was prepared and introduced to the Canadian market in 1992 as a cosmetic liposome moisturizing cream under the trademark LIPOBASE™. This scale-up operation for lower amounts of flavonoids or cannabinoids was also in evidence from Example 1 of the abovementioned patent where ˜0.2% of the flavonoid EGCG was readily encapsulated within the lipid bilayers of multilamellar vesicles.

### SUMMARY OF THE INVENTION

The ability to optimize lipophilic polyphenolic ingredients into liposomes was initially achieved by introducing an additional step into the original processing operation cited in U.S. Pat. No. 9,775,789. The initial investigation focused on EGCG whose room temperature solubility is greatly increased in the presence of combined equal amounts of alcohol or glycols and a citrate buffer solution (International Patent Publication no. 2008/004774 A1). In this patent publication, various alcohols and glycols combined with an equal portion of aqueous citrate buffer solution (100 mM, pH 5.0) were evaluated under autoclaving conditions to convert EGCG into its epimer GCG. EGCG solubilities in two of these solvent-buffer solutions were 44.9% for propylene glycol/buffer and 62.8% for the ethanol/buffer combination. The latter was excluded in their experiments due to the high temperatures required in autoclaving operations but we believe its usage in encapsulation of 0.5% EGCG is made possible by mixing, heating and dissolving a portion of it as usual, e.g., 0.2%, in a portion of the alcohol used for the phospholipid-EGCG-cholesterol phase. Ceramide was removed from the formulation and only minimal cholesterol used in order to increase encapsulation space in the lipid bilayers. In a separate container, the remaining 0.3% EGCG portion is easily dissolved with heating and mixing in the remaining required amount of alcohol from the 50% alcohol/citrate buffer. This latter step is then added to the alcohol-flavonoid-lipid phase resulting in a dark red translucent solution just prior to the hot aqueous phase addition. The heated aqueous phase consisting of purified water is added dropwise manually to the hot solvent-lipid-EGCG phase up to a period of time in which extensive thickening of the composition occurs and a possible need for greater input from the propeller stirring assembly. In a publication devoted to proliposome formulations, Williams et al (Journal of Pharmacy & Pharmacology 1991 43:154-161) attributed this thickening phase to precipitation of highly-stacked lipid bilayers which eventually convert into multilamellar vesicles on further water addition. The final result is a pink creamy dispersion that can be easily blended into a pharmaceutically acceptable gel, cream or lotion bases.

Buffer addition to the above formulation proved to be a much more significant contribution to the encapsulation process than simply providing EGCG solubilization enhancement. Sodium ion (Na⁺), and other alkali metal ions such as calcium (Ca²⁺) and magnesium (Mg²⁺), were found to play a significant role in the drug encapsulation process. Mobbili et al (Molecules 2018 23,441:1-18) utilized combined in silico and experimental methods to study the ability of neutral and anionic vesicles to encapsulate EGCG in the presence of both calcium and magnesium chlorides. The experimental procedure, a reverse phase evaporation or REV method, was a modification of the thin film hydration procedure. Phospholipids used were palmitoyl oleoyl phosphatidylcholine (POPC) and dioleoyl-sn-glycero-3-phosphoethanolamine (DOPE) plus cholesterol to produce neutral vescicles and replaced by cholesteryl hemisuccinate to produce anionic vesicles. The resulting liposomes contained an EGCG:phospholipid ratio of 1 to 6.5 and were present in the 0.5-5 μm size range. Results demonstrated that calcium ions hindered insertion of EGCG molecules into the liposome bilayer whereas magnesium ions significantly improved their bilayer insertion. Encapsulation efficiencies improved significantly in the presence of Mg⁺² to a maximum of 98.9% in anionic vesicles and only 82.2% in neutral vesicles whereas significant decreases occurred with Ca⁺². An additional feature was greater membrane thickness values expressed by use of the magnesium salt. The authors attribute the differences in encapsulation performances to the strong interaction of calcium ions to the negatively charged phosphate groupings of the phospholipids.

The aforesaid and other objectives of the present invention are realized by generally providing topical liposomal polyphenol compositions to be dispersed in aqueous pharmaceutically acceptable bases for treating and preventing various skin disorders.

According to an aspect of the invention, it is provided liposomal polyphenol compositions containing one or more lipophilic glycoside free flavonoids or one or more lipophilic glycoside free cannabinoids each polyphenol separately and optimally encapsulated within membrane lipid bilayers of mostly spherical vesicles.

According to another aspect of the invention, the liposomal polyphenol compositions comprise one or more glycoside free and high purity lipophilic flavonoids or cannabinoids chosen from plant or synthetic sources.

According to one more aspect of the invention, the liposomal polyphenol compositions contain mostly spherical bilayered vesicles comprised of one or more polyphenols, one or more glycerophospholipids, cholesterol, one or more cosmetically acceptable solvents and an aqueous solution.

According to another aspect of the present invention, it is provided a method for preparing liposomal polyphenol compositions containing a therapeutically effective amount of a glycoside free and lipophilic flavonoid or cannabinoid, optimally encapsulated within membrane lipid bilayers of spherical multilamellar vesicles.

According to yet another aspect of the invention, it is provided an aqueous pharmaceutically acceptable base in the form of a cream, lotion, gel or suspension having an acidic pH between 4.0 and 5.0 and comprising a liposomal polyphenol composition as prepared by the method disclosed herein.

According to a further related aspect of the invention, it is provided a final composition comprising a liposomal polyphenol composition for use in treating and preventing various skin disorders.

The present invention fulfills these needs and also other needs which will be apparent to those skilled in the art upon reading the following specifications.

### BRIEF DESCRIPTION OF THE DRAWINGS

The above and other objects, features and advantages of the invention will become more readily apparent from the following description, reference being made to the accompanying drawings in which:

FIG. 1 is a photograph depicting a baseline of a patient's back acne.

FIG. 2 is another photograph of the patient's back acne of FIG. 1.

FIG. 3 is a photograph depicting the treatment of a patient's back acne one week following the application of a liposomal polyphenol composition in accordance with an embodiment of the present invention.

FIG. 4 is another photograph of the patient's back acne of FIG. 3.

FIG. 5 is a photograph depicting the treatment of a patient's back acne two weeks following the application of a liposomal polyphenol composition in accordance with an embodiment of the present invention.

FIG. 6 is another photograph of the patient's back acne of FIG. 5.

FIG. 7 is a microscopic image of multilamellar lipid vesicles as used in a topical liposome polyphenol composition in accordance with an embodiment of the present invention.

### DETAILED DESCRIPTION OF A PREFERRED EMBODIMENT

Novel topical liposomal polyphenol compositions for treating and preventing various skin disorders will be described hereinafter. Although the invention is described in terms of specific illustrative embodiment(s), it is to be understood that the embodiment(s) described herein are by way of example only and that the scope of the invention is not intended to be limited thereby.

According to an embodiment of the invention, liposomal polyphenol compositions comprised of flavonoid and cannabinoid dispersed in aqueous pharmaceutically acceptable bases are provided. Such compositions comprise mostly spherical bilayer vesicles which comprise glycerophospholipids, lipophilic flavonoids or lipophilic cannabinoids, cholesterol, pharmaceutically acceptable solvents and an aqueous solution.

In some embodiments, the final composition comprises between about 15% to about 40% of the flavonoid or cannabinoid composition. Preferably, the final composition comprises between about 20% to about 30% of the liposomal flavonoid or cannabinoid composition (all weights given herein are by percentages unless otherwise specified).

In yet other embodiments, the bilayer vesicles are mostly spherical in shape. The spherical bilayer vesicles of the liposomal polyphenol compositions are unilamellar, oligolamellar or multilamellar, more preferably are multilamellar vesicles possessing diameters of about 2.5 μm or 7.5 μm size groupings depending on the molar mass of polyphenol used in the experiment.

In some embodiments, the bilayer vesicles comprise glycerophospholipids which may originate from natural or synthetic sources. Typically, the glycerophospholipids are chosen from egg or soy lecithin, and more preferably from soy lecithin.

In other embodiments, the glycerophospholipids may be chosen from one or more of phosphatidylcholine, phosphatidylglycerol, phosphatidylserine, phosphatidylinositol, phosphatidic acid, phosphatidylethanolamine. Typically, the glycerophospholipid is chosen from phosphatidylcholine, and more preferably is a phosphatidylcholine with fatty acid side chains comprised of 14 to 18 carbon atoms, or mixtures thereof. The final composition may comprise between about 1% to about 4% of glycerophospholipids, and more preferably between about 1.5% and about 3.0%.

In further embodiments, the final composition comprises from about 0.1% to about 1% of the lipophilic polyphenol. In such embodiments, the lipophilic, glycoside free and high purity polyphenol may be selected from natural or synthetic sources. Preferably, the lipophilic polyphenol may be selected from plant, fruit, vegetable, industrial hemp and synthetic sources and may include flavones, flavonols, flavanones, flavanols, isoflavones, anthocyanidins and cannabinoids. In yet other embodiments, the lipophilic polyphenols may each have molar masses ranging from about 200 to about 600 grams per mole, preferably about 300 grams per mole category.

In other embodiments, the final composition comprises between about 0.001% to about 0.025% cholesterol.

The pharmaceutically-acceptable solvents may be selected from alcohols and glycols, more preferably ethyl alcohol and propylene glycol or mixtures thereof. In such embodiment, the final composition generally comprises from about 0.5% to about 4% of each solvent usage.

In some embodiments, the aqueous solution is selected from acidified alkal metal salt solutions comprised of alkali metal cations sodium, potassium, magnesium or calcium and inorganic or organic acid anions chloride, sulfate, phosphate, acetate or citrate. In a preferred embodiment, the acidified alkali metal salt solutions are selected from magnesium salts dissolved in dilute citric acid solutions with pH values ranging between 3.0 to 4.0. Preferably, the final composition comprises between about 15% and about 40% of the aqueous solution.

According to another embodiment of the invention there is provided methods for preparing liposomal polyphenol compositions to be blended into pharmaceutically acceptable bases.

The final topical composition generally comprises batch sizes in the range of about 15% to about 40% of liposomal compositions, more preferably about 30%.

In most embodiments, batches comprise two-step processes. Glycerophospholipid, polyphenol, cholesterol and pharmaceutically acceptable solvent are stirred and heated until dissolved in a suitable container. Preferably the temperature of the heating phase is about 65° C. In a separate container unheated aqueous solution is initially added in small portions to the continuously heated lipid solvent phase with continuous stirring up to and through a thickening phase. In most embodiments, the transferring is done using a peristaltic pump. Propeller stirring speed may need adjustment at times to ensure complete movement of the formulation mass. As soon as a thinning phase appears in the first container, the method may further comprise adding the remaining aqueous solution to the first container at a more rapid rate. The process further comprises continuously stirring through to a final pourable dispersion phase.

In other embodiments, optimal encapsulation efficiencies of the polyphenolic ingredients are dependent on rigorous attention to phospholipid, solvent and aqueous media concentrations to be used in the formulation. With increasing polyphenolic ingredient content, sufficient solvent is necessary to easily dissolve the combined polyphenol, phospholipid and cholesterol within a reasonable time frame. Similarly, with increasing phospholipid concentrations, sufficient aqueous solution must be available to ultimately obtain a dispersion to meet a pourable lotion like consistency. Any scoop requirement of a dispersion in a production order may be more labor intensive for processing personnel in a pharmaceutical manufacturing department.

The method may further comprise storing the resulting liposomal polyphenol dispersion in a well-sealed container, and stored under nitrogen blanketing if an extensive delay were to occur in further processing operations.

In yet some embodiments, the liposomal polyphenol compositions may be blended into pharmaceutically acceptable bases such as aqueous gels, creams, lotions and suspensions. Such bases generally possess a pH range between 4.0 and 5.0 and typically comprise conventional non-medicinal ingredients required to perform formulation functions familiar to those skilled in the art.

In some embodiments, the final composition is used for helping to treat and prevent reddened, itchy and dry skin conditions by administering a therapeutically effective amount of the final composition disclosed herein to a subject in need thereof, including but not limited to, solar radiation, insect stings and atmospheric pollutants comprising reactive oxygen species.

In other embodiments, the final composition may also be used for treating various inflammatory dermatoses by administering a therapeutically effective amount of the final composition disclosed herein to a subject in need thereof, including, but not limited to, eczema, allergic dermatitis, contact dermatitis, aural eczematoid dermatitis, otitis externa, acne, hyperpigmentation, melasma, rosacea, and also conditions arising as a result of genetic, immune or neuropathic disease such as sarcoidosis, scleroderma, mastocytosis, postherpetic neuralgia, epidermolysis bullosa, pyoderma gangrenosum and neuropathic pain.

### EXAMPLES

These examples are illustrative of the wide range of applicability of the present invention and is not intended to limit its scope. The liposomal compositions may be readily adapted for use as optic and oral preparations. Modifications and variations of the topical compositions can be made therein without departing from scope of the present invention. Although any method and material similar or equivalent to those described herein can be used in the practice for testing of the present invention, the preferred methods and materials are described. Any suitable pharmaceutical bases known in the art may be used within the scope of the present invention.

The following polyphenolic examples were prepared in 200-300 gram batch sizes. Equipment utilized in the experiments were the following: Fisherbrand Isotemp Digital Hotplate Stirrer; Caframo Compact Digital Stirrer; Gilson Minipuls 3 peristaltic pump; Nikon Eclipse TS-100 400× microscope; Biochrom Ultrospec 2000 UVNisible spectrophotometer; Fisher Scientific accuSpin Micro 17R centrifuge, NDJ-5S Viscometer #212N2040. Encapsulation efficiencies (EE) were performed internally on the supernatant component of centrifuged samples using conventional UV spectrophotometric assay procedures.

### Example 1

### Liposomal 0.5% EGCG Dispersion (Na+)—Manual Operation

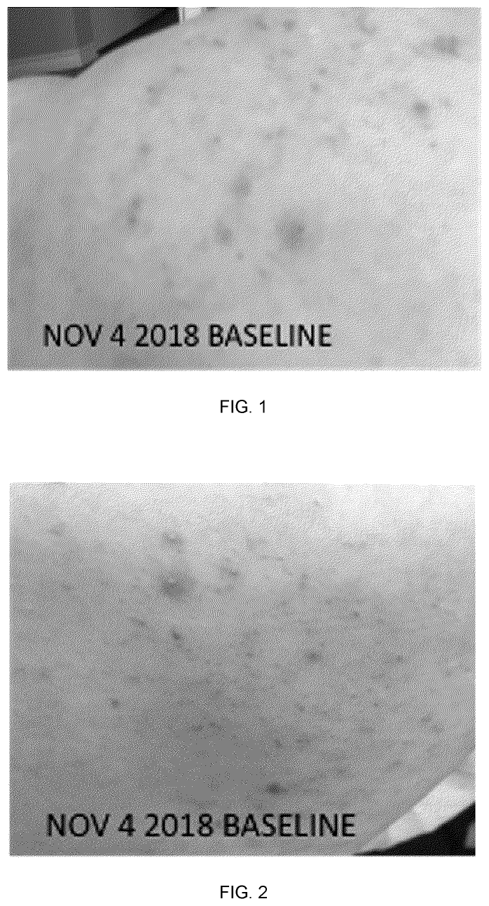

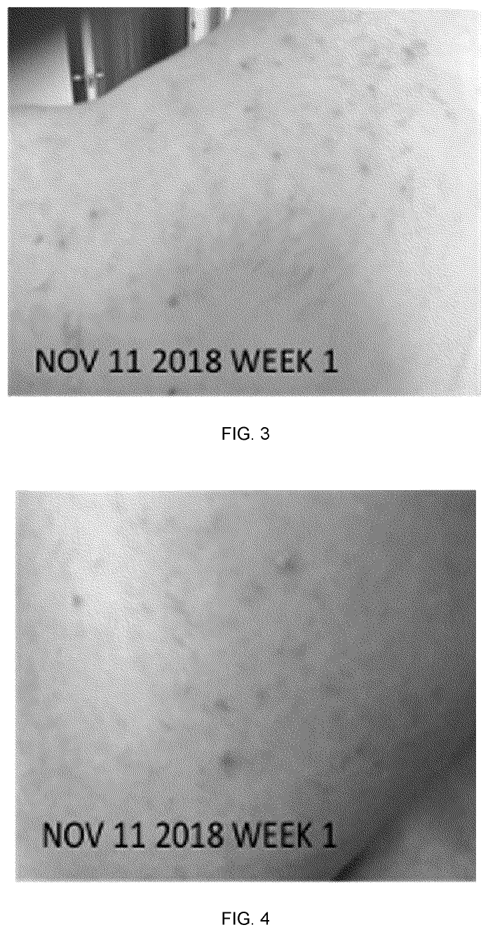

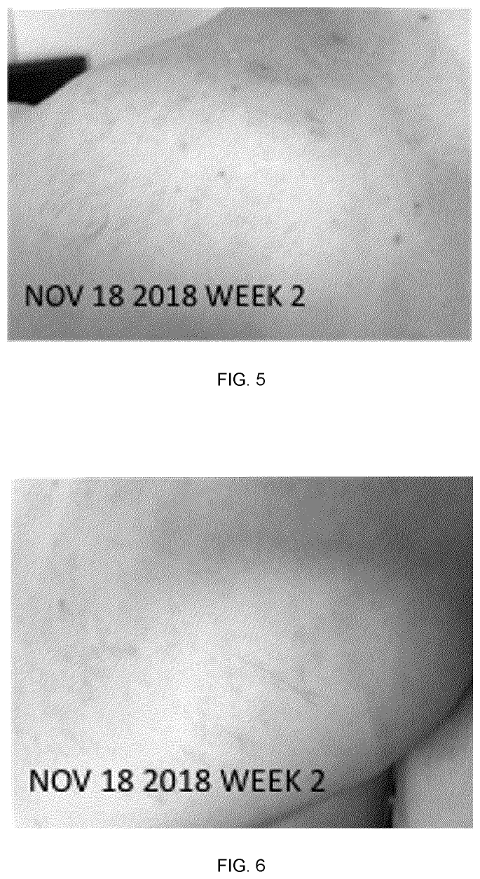

This example is from prior lab experiments and the actual dispersion used in a soothing base cream (Example 10) in 2018 to treat an individual with back acne (as shown in FIGS. 1 to 6). In this example, the alkali metal sodium ion was utilized in the formulation as a 100 mM sodium citrate buffered component of the lipid phase. The ratio of EGCG to HPC was 1 to 4.5 and the dispersion concentration represented 15% of a final composition. Ingredients listed in PART 1 of the following table were heated and stirred under good agitation at a temperature of about 65° C. until a dark red transparent solution was obtained. PART 2 ingredients were heated and manually stirred until dissolved and then added to PART 1 container with continuous stirring. Heated purified water from PART 3 was then manually added dropwise with the aid of a pipette to heated PART 1 ingredients until occurrence of and throughout a thickening phase. Following a short-term thickening phase, the aqueous solution was then added at a more rapid rate with continuous stirring throughout the thinning phase to the end of the procedure. Stirring was continued for an additional 20 minutes and the resulting creamy pink dispersion stored in a well-sealed container.

PART 1

| INGREDIENTS | % CONC. | % CREAM |
|---|---|---|
| *Phospholipon 90H ™ | 15.000 | 2.250 |
| Cholesterol USP | 0.080 | 0.012 |
| Ceramide 2 | 0.020 | 0.003 |
| **EGCG | 1.773 | 0.225 |
| Alcohol Anhydrous | 15.333 | 2.300 |
| SUB-TOTAL | 32.207 | 4.790 |

PART 2

| | | |
|---|---|---|
| Sunphenon EGCG | 2.173 | 0.275 |
| Na Citrate Buffer 100 mM pH 5.0 | 4.667 | 0.700 |
| Alcohol Anhydrous | 4.667 | 0.700 |
| SUB-TOTAL | 11.507 | 1.675 |

PART 3

| | | |
|---|---|---|
| Purified Water | 56.286 | 8.535 |
| TOTAL | 100.000 | 15.000 |

*Hydrogenated phosphatidyl choline (HPC), Lipoid LLC, Newark NJ
**Epigallocatechin gallate, Taiyo Intl. Inc, Minneapolis MN

This formulation resulted in a moderately thick lotion in consistency but was still pourable. Values appearing for EGCG and other polyphenolic ingredients in % concentration columns have been adjusted to compensate for actual assay data provided by suppliers.

### Example 2

### Liposomal 0.5% EGCG Dispersion (Na+)—Peristaltic Pump Operation

This formulation is a recent repeat of Example 1 with the following minor ingredient changes: namely, removing ceramide 2 and doubling cholesterol content. An additional change included converting the manual aqueous dropwise addition to a peristaltic pump processing operation which is adopted for all future preparations. In this experiment, the aqueous addition commenced at 0.5 mL/min for 24 minutes up to a thickening phase, mixing with increased stirring speed for 10 minutes to ensure complete movement of formulation mass, changing addition rate at 1.3 mL/min for 7 minutes and the final rate at 6 mL/min to the end of the operation.

PART 1

| INGREDIENTS | % CREAM | % CONC. |
|---|---|---|
| Phospholipon 90H ™ | 2.250 | 15.000 |
| Cholesterol USP | 0.023 | 0.153 |
| EGCG | 0.225 | 1.627 |
| Alcohol Anhydrous | 2.300 | 15.333 |
| SUB-TOTAL | 4.798 | 32.113 |

PART 2

| | | |
|---|---|---|
| Sunphenon EGCG | 0.275 | 1.993 |
| Na Citrate Buffer 100 mM pH 5.0 | 0.700 | 4.667 |
| Alcohol Anhydrous | 0.700 | 4.667 |
| SUB-TOTAL | 1.675 | 11.327 |

PART 3

| | | |
|---|---|---|
| Purified Water | 8.527 | 56.560 |
| TOTAL | 15.000 | 100.000 |

Similar to the light pink dispersion of Example 1, this formulation was a moderately thick lotion in consistency but was still pourable.

### Example 3

### Encapsulation Efficiency Determination

Unentrapped polyphenols were separated by centrifugation from the following dispersions and their concentrations in the resulting supernatants determined spectrophotometrically at applicable wavelengths. Those dispersions containing polyphenols with molar masses in the 300 g/M category possessed liposomes half the size of those in the >400 g/M and in far superior numbers. Combined with a mild gelling contribution of MgCl2 to these low density vesicles, centrifugation required addition of 0.1M sodium hydroxide to convert the magnesium salt into the readily pelletable magnesium hydroxide. This adjustment was not necessary for the >400 g/M sample. Blank solutions comprised of the same solvent combinations and aqueous solutions were then prepared and utilized to construct calibration curves by plotting relative absorbance values versus polyphenol concentrations ranging between 1 and 12 mg/ml. The concentration of the unknown can then be determined from the following equation:

$$\% EE = ( \frac{Total Amount Loaded - Amount in Supernatant}{Total Amount Loaded} ) \times 100$$

### Example 4

### Liposomal 0.5% EGCG Dispersions (Mg++)—Optimal Encapsulation

In this and subsequent examples, the formulations are reduced from a three part to a two part processing operation. The alkali metal magnesium ion replaces sodium ion and is included as magnesium chloride at a similar molar concentration (0.1M) dissolved in dilute citric acid solution. The latter solution replaces purified water in the aqueous phase component of the formulation. Ingredients listed in PART 1 of the table below are heated and stirred under good agitation at a temperature about 65° C. until complete dissolution. The unheated aqueous solution listed in PART 2 is then added dropwise with the aid of a peristaltic pump at a rate of about 10 mL/min to the heated PART 1 ingredients until occurrence of and throughout a thickening phase. Propeller stirring speed is adjusted to ensure continuous movement of the formulation mass. Following this short-term thickening phase, the aqueous solution is then added at a more rapid rate of about 20 mL/min throughout the thinning phase of the stirred dispersion. Stirring is continued for an additional 20 minutes and the resulting homogeneous dispersions stored in well-sealed containers.

PART 1

| | % CREAM | % CONC. | % CREAM | % CONC. | % CREAM | % CONC. |
|---|---|---|---|---|---|---|
| INGREDIENTS | A | A | B | B | C | C |
| Phospholipon 90H ™ | 2.250 | 11.250 | 2.750 | 9.167 | 3.250 | 8.125 |
| Cholesterol USP | 0.023 | 0.113 | 0.023 | 0.075 | 0.023 | 0.056 |
| EGCG | 0.500 | 2.715 | 0.500 | 1.810 | 0.500 | 1.358 |
| Propylene Glycol | 0.700 | 3.500 | 1.500 | 5.000 | 2.000 | 5.000 |
| Alcohol Anhydrous | 3.000 | 15.000 | 3.000 | 10.000 | 3.500 | 8.750 |
| SUB-TOTAL | 6.473 | 32.578 | 7.773 | 26.052 | 9.273 | 23.289 |

PART 2

| | | | | | | |
|---|---|---|---|---|---|---|
| 0.1M MgCl2 in 0.1% CA | 13.527 | 67.423 | 22.227 | 73.948 | 30.727 | 76.711 |
| TOTAL | 20.000 | 100.000 | 30.000 | 100.000 | 40.000 | 100.00 |
| EE % | | 67.9 | | 85.6 | | 84.2 |
| Size Range μm | | 5-7.5 | | 5-7.5 | | 5-7.5 |
| Viscosity cP | | 118 | | 20.6 | | 25.3 |

Dispersion A was prepared using an EGCG:HPC ratio of 1:4.5 and a 20% dispersion content. The EE data also reveals that the encapsulating process is impeded during a more sustained thickening phase and due to insufficient quantities of both phospholipid and aqueous medium to accommodate optimal EGCG encapsulation.

Dispersion B formulation was changed from the EGCG:HPC ratio of 1:4.5 to 1:5.5 and the dispersion content raised from 20% to 30%. The dispersion provided an excellent encapsulation efficiency of 85.6%.

Dispersion C formulation was changed to increase the EGCG:HPC ratio to 1:6.5 and the dispersion content to 40% in order to provide additional phospholipid for EGCG encapsulation. The EE result indicates that an optimal encapsulation appears to be achieved by dispersion B and a value slightly superior to the maximum value 82.3% achieved by Mobbili et al for EGCG encapsulation using MgCl2 and neutral phospholipids like HPC. A larger and more homogeneous size range is also apparent with our liposomes. A contributing factor may be due to the greater aqueous solubility of EGCG versus other polyphenols. However the Mobbili group were able to achieve 100% EE with the anion vesicles.

### Example 5

### Liposomal 0.5% CBD Dispersion (Mg++)—Optimal Encapsulation:

The following experiments are an extension of Example 4 using cannabidiol (CBD). The formulations were prepared with 30% and 40% dispersions and the same CBD:HPC ratio of 1:5.5 and processing operations used in Example 4.

PART 1

| | % CREAM | % CONC | % CREAM | % CONC |
|---|---|---|---|---|
| INGREDIENTS | A | A | B | B |
| Phospholipon | 2.750 | 9.167 | 2.750 | 6.875 |
| 90H ™ | | | | |
| Cholesterol USP | 0.023 | 0.077 | 0.023 | 0.058 |
| *Cannabidiol | 0.500 | 1.667 | 0.500 | 1.250 |
| Propylene Glycol | 1.500 | 5.000 | 1.500 | 3.750 |
| Alcohol | 3.000 | 10.000 | 3.000 | 7.500 |
| SUB-TOTAL | 7.773 | 25.910 | 7.773 | 19.433 |

PART 2

| | | | | |
|---|---|---|---|---|
| 0.1M MgCl in 0.1% citric acid | 22.227 | 74.090 | 32.227 | 80.567 |
| TOTAL | 30.000 | 100.000 | 40.000 | 100.000 |
| Encapsulation Efficiency % | | 92.8 | | 98.5 |
| Particle Size Range μm | | 1-5 | | 1-5 |
| Viscosity cP | | 117.4 | | <20 |

*Global Cannabinoids, Las Vegas, Nevada

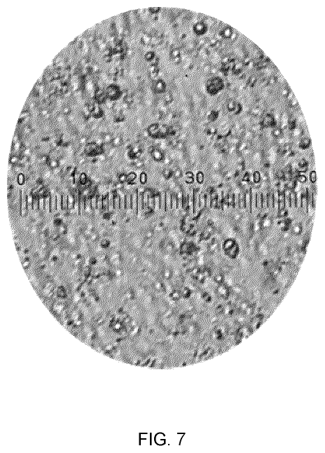

Results differed from those of the EGCG experiments in that an optimal encapsulation efficiency of 98.5% occurred for the 40% dispersion. Particle size evaluation by microscopic examination following dilution of 1 part dispersion to 20 parts purified water revealed mostly spherical multilamellar vesicles for both formulations. Vesicle diameter sizes were estimated in the 1-5 μm range with most appearing in the 2.5 μm grouping (as shown in FIG. 7). The significantly greater vesicle numbers and smaller particle size values observed microscopically in the CBD dispersions versus their EGCG counterparts is obviously impacted by smaller spatial area requirements for CBD in the vesicle membrane lipid bilayers. This spatial area requirement is dependent on a molecule's molar mass, for example only 314 g/M for CBD versus 458 g/M for EGCG. Vesicle density differences and the gelling contribution of magnesium chloride also contributed to problems encountered in centrifuging CBD samples for determining encapsulation efficiency.

### Example 6

### Effect of Altering Mg++ Content on CBD Optimal Encapsulation:

The following experiment is an extension of Example 5 to ascertain the effect of reducing the amount of MgCl2 on the optimal EE of CBD. The formulation was prepared with the same 40% dispersion B replacing only half the amount of MgCl2 solution with an equal portion of purified water. The rate of addition of the aqueous systems remained unchanged, e.g., the salt solution was added at the 10 ml/min rate through the thickening phase and the purified water at 20 ml/min through the thinning phase to the end.

PART 1

| INGREDIENTS | % CREAM A | % CONC A |
|---|---|---|
| Phospholipon 90H ™ | 2.750 | 6.875 |
| Cholesterol USP | 0.023 | 0.058 |
| *Cannabidiol | 0.500 | 1.250 |
| Propylene Glycol | 1.500 | 3.750 |
| Alcohol | 3.000 | 7.500 |
| SUB-TOTAL | 7.773 | 19.433 |

PART 2

| | | |
|---|---|---|
| 0.1M MgCl in 0.1% citric acid | 16.113 | 40.283 |
| Purified Water | 16.112 | 40.284 |
| TOTAL | 40.000 | 100.000 |
| Encapsulation Efficiency % | | 85.6 |
| Particle Size Range μm | | 1-5 |
| Viscosity cP | | 20.6 |

The results show a dramatic change in EE from 98.2% to 85.6%. Such an EE change due to a drop in the aqueous salt concentration may explain the unanticipated EE behaviour of the EGCG formulations in Example 4. It is worth noting that Mobbili and co-authors recognized the contribution of different MgCl2 concentrations on EE of EGCG formulations.

### Example 7

### Liposomal 0.25% CBD+0.25% THC Dispersion (Mg++)

This example contains an additional cannabinoid 0.25% tetrahydrocannabinol (THC) and half the amount of CBD used in Example 4. In preparing the THC for inclusion in the formulation, the exact amount of the heptane/isopropanol solvent combination containing THC (138 mg/ml) was transferred to an appropriate container, the solvents removed under a steady stream of nitrogen and the resulting residue dissolved with gentle warming in portions of the alcohol to be used in the formulation. Processing operations were identical to those carried out in Example 3.

PART 1

| INGREDIENTS | % CREAM | % CONC. |
|---|---|---|
| Phospholipon 90H ™ | 2.750 | 6.875 |
| Cholesterol USP | 0.023 | 0.058 |
| Cannabidiol | 0.250 | 0.625 |
| *THC | 0.250 | 0.625 |
| Propylene Glycol | 1.500 | 3.750 |
| Alcohol | 3.000 | 7.500 |
| SUB-TOTAL | 7.773 | 19.433 |

PART 2

| | | |
|---|---|---|
| 0.1M MgCl2 in 0.1% citric acid | 32.227 | 80.567 |
| TOTAL | 40.000 | 100.000 |
| Encapsulation Efficiency % | | ND |
| Particle Size Range μm | | 2.0-5 |
| Viscosity cP | | <20 |

*Rhodes Technologies, Coventry RI

Results demonstrated that the pearly white dispersion possessed the attributes of a low viscosity lotion. Encapsulation efficiency was not performed on this dispersion but the value should be comparable to that of CBD in Example 5. Particle size estimation by microscopic examination revealed mostly spherical multilamellar vesicles for the dispersion in the 2.0-5 μm range with most appearing in the lower 2.5 μm grouping.

### Example 8

### Liposomal 0.5% Quercetin Dispersion (Mg++)

This experiment utilizes anhydrous quercetin and is prepared according to the same processing operations outlined in Example 3.

PART 1

| INGREDIENTS | % CREAM | % CONC. |
|---|---|---|
| Phospholipon 90H ™ | 2.750 | 6.875 |
| Cholesterol USP | 0.023 | 0.058 |
| *Quercetin Anhydrous | 0.500 | 1.250 |
| Propylene Glycol | 1.500 | 3.750 |
| Alcohol Anhydrous | 3.000 | 7.500 |
| SUB-TOTAL | 7.773 | 19.433 |

PART 2

| | | |
|---|---|---|
| 0.1M MgCl2 in 0.1% citric acid | 32.227 | 80.567 |
| TOTAL | 40.000 | 100.000 |
| Encapsulation Efficiency % | | 94 |
| Particle Size Range μm | | 1-5 |
| Viscosity cP | | 113 |

*TIMES, Durachem Distribution, Beaconsfield Quebec Canada

Results were consistent with the 40% dispersion data for CBD in Example 4 demonstrating an excellent encapsulation efficiency of 94%. Particle size estimations by microscopic examination revealed mostly spherical multilamellar vesicles for the dispersions in the 1-5 μm range with most appearing in the 2.5 μm grouping again comparable to that of the CBD vesicles. The viscosity for the 40% yellow dispersion was slightly greater than its CBD counterpart. This viscosity difference may again be due to their molar mass differences, e.g., quercetin 302 g/M versus cannabidiol 314 g/M where greater numbers of MLVs are anticipated with quercetin resulting in increased viscosity.

### Example 9

### Liposomal 0.75% Quercetin Dispersion (Mg++)

This experiment is an extension of Example 7 to determine pourability limit of a dispersion with a greater polyphenol concentration. Several changes include the use of quercetin dihydrate instead of the anhydrous form, lower QC:HPC ratio 1:3.7, 30% dispersion and reversal of the solvents and their contents.

PART 1

| INGREDIENTS | % CREAM | % CONC. |
|---|---|---|
| Phospholipon 90H ™ | 2.800 | 9.333 |
| Cholesterol USP | 0.023 | 0.077 |
| *Quercetin Dihydrate | 0.750 | 2.860 |
| Propylene Glycol | 3.500 | 11.667 |
| Alcohol Anhydrous | 1.500 | 5.000 |
| SUB-TOTAL | 8.573 | 28.937 |

PART 2

| | | |
|---|---|---|
| 0.1M MgCl2 in 0.1% citric acid | 21.427 | 71.063 |
| TOTAL | 30.000 | 100.000 |
| Encapsulation Efficiency % | | ND |
| Particle Size Range μm | | ND |
| Viscosity cP | | 8,928 |

*TIMES, Durachem Distribution, Beaconsfield Quebec Canada

Results revealed that the leading solvent propylene glycol created small bubbles throughout the initial ingredient heating process but had no effect on the final formulation. The yellow dispersion easily met pourability requirements and further improvements are expected to be met with a 40% dispersion.

### Example 10

### Soothing Cream Base for a 15% Liposomal Dispersion

The following formula contains ingredients for an 85% soothing cream base plus a 15% liposomal polyphenol composition as exemplified in Examples 1 and 2. Ingredients 1 through 8 are added sequentially with good agitation in a suitable container equipped with propellor stirring. Adjustment of pH between 4.0 to 5.0 is conducted if necessary by adding either 10% citric acid or sodium citrate dihydrate solutions. Items 9 and 10 are then added in sequence with good agitation and stirring continued for an additional twenty minutes.

| NO. | INGREDIENTS | % CREAM |
|---|---|---|
| 1 | Purified Water | 74.200 |
| 2 | Sorbo 70% | 4.000 |
| 3 | Disodium EDTA | 0.100 |
| 4 | Phenoxyethanol | 0.680 |
| 5 | Citrus Extract | 0.120 |
| 6 | Cyclomethicone | 1.000 |
| 7 | Isopropyl Palmitate | 2.500 |
| 8 | Liposome Concentrate | 15.000 |
| 9 | Solagum Tara | 1.200 |
| 10 | Sepigel 305 | 1.200 |
| | TOTAL | 100.000 |

### Example 11

### Back Acne Treatment

A prototype of Example 8 was used to evaluate a proof-of-concept experiment in treating acne on the back of a 30-year old adult male with twice daily applications in morning and evening. Acne back grading was estimated between 2.0 and 3.0 utilizing The Leeds Revised Acne Grading System. The individual agreed to test the formulation for a two week period instead of an already dermatologist prescribed and patient purchased anti-acne product (Clindoxyl® Gel, Stiefel). Two back areas exhibiting greater numbers and more severe lesions (upper back left shoulder & lower right back) were selected for daily treatment and weekly follow-up by camera recordings. Baseline, 1 & 2-week recordings (as shown in FIGS. 1 to 6) demonstrated significant changes after only 1-week treatment and virtually complete clearance at end of the second week. Treatment stopped with the individual completely satisified with the results of the treatment. Similar treatment performance for more severe acne conditions is anticipated with an optimized liposomal encapsulated EGCG cream, i.e. >85% EE.

While illustrative and presently preferred embodiment(s) of the invention have been described in detail hereinabove, it is to be understood that the inventive concepts may be otherwise variously embodied and employed and that the appended claims are intended to be construed to include such variations except insofar as limited by the prior art.

1) A liposomal polyphenol composition for topical administration to a subject in need thereof, comprising: one or more lipophilic, glycoside free and high purity polyphenol ingredients encapsulated within lipid membrane bilayers of spherical vesicles; wherein the lipid membrane bilayers of spherical vesicles comprise glycerophospholipids, cholesterol, pharmaceutically acceptable solvents and an aqueous medium.

2) The liposomal polyphenol composition of claim 1, wherein the one or more lipophilic and glycoside free polyphenols are flavonoids derived from various plants or synthetic processes and are selected from high purity flavones, flavonols, flavanones, anthocyanidins, isoflavones and flavanols.

3) The liposomal polyphenol composition of claim 1, wherein the one or more lipophilic and glycoside free polyphenols are high purity cannabinoids derived from industrial hemp and synthetic processes.

4) The liposomal polyphenol composition of claim 1, wherein each lipophilic and glycoside free polyphenol has molar mass values ranging between about 200 grams per mole to about 600 grams per mole.

5) The liposomal polyphenol composition of claim 1, wherein each lipophilic and glycoside free polyphenol is optimally encapsulated within the lipid membrane bilayers of mostly spherical vesicles possessing mean diameter sizes:
a) about 2.5 m for encapsulated polyphenols in the lower molar mass category including quercetin and cannabidiol with molar masses of 302 and 314 grams per mole respectively; and
b) about 5 m for encapsulated polyphenols in the upper molar mass category including EGCG with a molar mass of 458 grams per mole.

6) The liposomal polyphenol composition of claim 1, wherein the one or more glycerophospholipid is selected from phosphatidylcholine, phosphatidylglycerol, phosphatidylethanolamine, phosphatidylserine, phosphatidic acid, phosphatidylinositol, and a mixture thereof.

7) The composition of claim 6, wherein the one or more glycerophospholipid is a phosphatidylcholine comprising saturated fatty acid side chains having between 14 to 18 carbon atoms, or mixtures thereof.

8) The liposomal polyphenol composition of claim 1, wherein the solvents are of pharmaceutical quality and selected from ethyl alcohol, propylene glycol, and mixtures thereof.

9) The liposomal polyphenol composition of claim 1, wherein the aqueous medium comprises an aqueous alkali metal salt dissolved in a mildly acidic aqueous solution.

10) The composition of claim 9, wherein the alkali metal salt comprises an alkali metal cation selected from sodium, magnesium, potassium, calcium and an inorganic or organic anion comprising chloride, sulfate, phosphate, acetate or citrate.

11) The composition of claim 9, wherein the alkali metal salt is magnesium chloride.

12) The composition of claim 9, wherein the acid of the mildly acidic aqueous solution is selected from acetic acid and citric acid.

13) (canceled)

14) A method for preparing the liposomal polyphenol compositions of claim 1 comprising final polyphenol concentrations between 0.1% and 1%, the method comprising:
a) heating, stirring and completely dissolving in a suitable container maintained at about 65° C., one or more polyphenolic ingredients, one or more glycerophospholipids, cholesterol and one or more pharmacopeal quality solvents.
b) adding the aqueous medium in the unheated form of an acidic alkali metal salt solution to the solution of step a) at a rate of or about 10 mL per minute with continuous stirring until occurrence of and throughout a thickening phase;
c) increasing the rate of addition to about 20 mL per minute at the onset of a thinning phase and to the end of the operation;
d) adjusting stirrer speed as required in order to maintain constant movement of the formulation mass;
e) continuing stirring after completion of aqueous medium addition until a homogeneous pourable dispersion is obtained.

15) The method as claimed in claim 14, wherein the aqueous medium in the form of an acidic alkali metal salt solution is added to the solution of step a) with the aid of one or more peristaltic pumps.

16) The method as claimed in claim 14, the method further comprising the step of storing the formulation mass in one or more sealed containers, under nitrogen if needed due to an excessive delay in a further processing requirement.

17) The method as claimed in claim 14, wherein the unheated aqueous solution is magnesium chloride dissolved in dilute citric acid solution.

18) The method as claimed in claim 14, wherein the dispersion pH is in the range of about 3.0 to about 4.0.

19) A final composition comprising a pharmaceutically acceptable base in the form of an aqueous cream, gel, lotion or suspension having a pH value between 4.0 and 5.0 and comprising a liposomal polyphenol composition as claimed in claim 1.

20) The final composition of claim 19 comprising a concentration between about 5% to about 40% of the liposomal polyphenol composition by weight of the final composition.

21) The final composition of claim 19 comprising a concentration between about 0.1% to about 1% of the one or more lipophilic flavonoids contributed by the liposomal polyphenol composition.

22) The final composition of claim 19 comprising a concentration between about 0.1% to about 1% of the one or more lipophilic cannabinoids contributed by the liposomal polyphenol composition.

23) The final composition of claim 19 comprising a concentration between about 1% to about 4% of the one or more phosphatidylcholines contributed by the liposomal polyphenol composition.

24) The final composition of claim 19 comprising a concentration between about 0.001% to about 0.025% of the cholesterol contributed by the liposomal polyphenol composition.

25) The final composition of claim 19 comprising a concentration between about 1% to about 4% of each solvent contributed by the liposomal polyphenol composition.

26) (canceled)

27) (canceled)

28) (canceled)

29) (canceled)

30) (canceled)

31) The use of the final composition of claim 19 for treating a skin disorder by administering a therapeutic amount of the composition to a human being in need thereof.

32) (canceled)

33) (canceled)
